(19) United States
(12) Patent Application Publication
(10) Pub. No.: US 2008/0139611 A1
(43) Pub. Date: Jun. 12, 2008

(54) METHODS OF USING 4-PHENYLAMINOQUINOLINES AS TOPICAL NON-STEROIDAL ANTIINFLAMMATORY COMPOUNDS

(76) Inventors: Jan Wasley, Guillford, CT (US); Jacob Plattner, Berkeley, CT (US)

Correspondence Address: GOODWIN PROCTER LLP;PATENT ADMINISTRATOR, EXCHANGE PLACE, BOSTON, MA, 02109-2881, US

(21) Appl. No.: 11/873,116

(22) Filed: Oct. 16, 2007

Related U.S. Application Data
(60) Provisional application No. 60/852,191, filed on Oct. 17, 2006.

(51) Int. Cl. A61K 31/4706; A61P 27/02; A61P 19/02; A61P 29/00; A61P 35/00

(52) U.S. Cl. 514/313

(57) ABSTRACT

A skin disorder, a disorder associated with pain, fever, or inflammation, a proliferative disorder, or an ocular disorder is treated by topically administering a pharmaceutically acceptable formulation containing a therapeutically effective amount of a 4-phenylaminoquinoline compound.

### CROSS-REFERENCE TO RELATED APPLICATION

The present applications claims priority to, and the benefits of, U.S. Provisional Application Ser. No. 60/852,191, filed on Oct. 17, 2006, the entire disclosure of which is hereby incorporated by reference.

### BACKGROUND OF THE INVENTION

In general, the invention relates to medical management of inflammatory and proliferative disorders.

The skin is the largest organ of the body, covering the entire outside of the body, and includes the epidermis, dermis, and subcutaneous layers. Numerous disorders of the skin are known, ranging from that which merely causes discomfort or psychological stress, such as rashes, to life-threatening conditions such as skin cancer.

Skin cancer is the most common form of cancer in the United States. Basal cell carcinoma develops from abnormal growth of the cells in the lowest layer of the epidermis and is the most common type of skin cancer. Squamous cell cancer involves changes in the squamous cells, found in the middle layer of the epidermis. Melanoma, which occurs in the melanocytes, is less common than squamous or basal cell carcinoma but is much more dangerous. Melanoma is the leading cause of death from skin disease.

Actinic keratosis is a precancerous skin growth usually caused by sun exposure, which may develop into squamous cell cancer. Actinic keratosis lesions are the most common neoplastic skin lesions detected in individuals with Fitzpatrick skin type I or II. Actinic keratosis lesions appear as papules (i.e., small, solid, elevated skin lesions) in a vast spectrum of sizes, shapes, colors, and other characteristics. Their size and shape can range from a well-circumscribed, single millimeter papule to an irregularly shaped lesion that can span several centimeters. These neoplasms can be flesh colored, red or pigmented and can also scale or become hyperkeratotic. The most common sites for these lesions are the face, ears, scalp, neck, forearms, and hands.

Psoriasis is a non-contagious, inflammatory disease of the skin that has been diagnosed in 4.5 million adults in the United States. The most common form, plaque psoriasis, appears as raised, red patches or lesions covered with a silvery white buildup of dead skin cells, called scale. About 10% to 30% of people with psoriasis also develop psoriatic arthritis. Psoriatic arthritis is a condition that causes swelling and pain in and around the joints and also affects tissues surrounding the joints, including tendons and ligaments. Skin inflammation is evident in this condition, particularly on the elbows, knees and scalp. There is no cure for psoriatic arthritis and treatment is designed to minimize pain and stiffness.

Eczema, also known as atopic dermatitis, is a skin condition that can affect all age groups. The severity of the condition varies. Mild cases of eczema result in dry, hot and itchy skin, whilst severe cases result in the skin becoming broken, raw and bleeding. Although the appearance of eczema can look unpleasant, the condition is not contagious. However, while the inflammation of eczema can be reduced with proper treatment, the skin will still be sensitive to flare-ups and require extra care.

The causes of eczema are varied and depend upon the type. Atopic eczema is thought to be a hereditary condition. It is proposed that people with atopic eczema have an abnormally heightened sensitivity to allergens in their environment. In atopy there is an excessive reaction by the immune system producing inflamed, irritated and sore skin. Associated atopic conditions include asthma and hay fever. Detergents, chemicals such as nickel, allergens such as yeast growths, or poor blood circulation in the legs may exacerbate other types of eczema. The causes of certain types of eczema remain to be explained, though links with environmental factors and stress are being explored.

Osteoarthritis (OA), also known as degenerative joint disease, is a chronic condition which is characterized by the breakdown of cartilage in the joints. Most commonly affected are the weight-bearing joints such as the hips, knees and lower back. Other joints that might be impacted by OA include those of the neck, small fingers, the base of the thumb and the big toe. Cartilage is a tissue that cushions the ends of the bones in joints enabling smooth and easy movement. The breakdown of cartilage causes a condition where the bones of the joint come in contact with each other and rub together during movement. This results in inflammatory symptoms of pain, stiffness and loss of motion of the joint. There are currently an estimated 21 million Americans who suffer from OA.

Rheumatoid arthritis (RA) is a chronic inflammatory disease that affects about 2.1 million Americans or about 1% of the population. It is characterized by inflammation of the lining, or synovium, of the joints and can result in long-term damage reflected in chronic pain, loss of function and disability. There are three stages of RA. The first stage involves a swelling of the synovial lining, which can cause redness, pain, stiffness, warmth and swelling around the joint. The second stage involves a rapid cellular proliferation, resulting in a thickening of the synovium. In the third stage, inflamed cells can release cytokines and enzymes that result in further inflammation and damage to bones and cartilage in the joints. This can cause additional pain, loss of movement and loss of shape and alignment of the joint.

Herpes simplex virus (HSV) commonly referred to as “herpes virus” or “herpes,” is an infectious disease which has reached crisis proportions nationally with estimated numbers of infected people at 70%-80% of our population as reported by the American Social Health Association (ASHA) and growing annually by 500,000 people or more. There are two common types of herpes: herpes simplex virus 1 (HSV 1) and herpes simplex virus 2 (HSV 2).

Herpes enters the human body through minuscule breaks in the epidermal tissue usually by contact with an infected host and is marked by eruption of one or more vesicles, usually in groups, following an incubation period of approximately four to ten days. Typically the course of the infectious outbreak initiates with the prodromal stage and advances to vesicular eruption, which is followed by ulceration, coalescing, resolution, and the latency period. The outbreak can last for several weeks and on average lasts two to three weeks, although in some immune-compromised individuals the outbreak can last for months. The vesicles can appear anywhere on the skin or mucosa, typically on the lips as cold sores, glands, oral mucosa, conjunctiva and cornea, genitalia, anal mucosa and peri-anal tissue.

Herpes symptoms include inguinal swelling, pain, fever, malaise, headaches, muscle aches, and swollen glands. Some individuals with trigeminal-nerve-affected oral herpes have excruciating facial pain, difficulty swallowing, eating and facial swelling. Individuals with the sacral-nerve-affected have severe upper leg pain, swelling, and great difficulty walking.

Dry eye is a condition wherein the relative amount of lachrymal fluid is below average or abnormal in quality, and can result with or without corneal and/or conjunctival lesions (Yamada, M. et al., Folia Ophthalmol. Jpn., 43:1289-1293, 1992). Examples of this condition include dry eye observed in hypolacrimation, alacrima, xerophthalmia, Sjogren syndrome, keratoconjunctivitis sicca, Stevens-Johnson syndrome, ocular pemphigoid, marginal blepharitis, diabetes and the like, dry eye observed after cataract operation, dry eye in conjunction with allergic conjunctivitis and the like, and dry eye due to hypolacrimation caused by increased visual display terminal (VDT) work, or an arid environment.

The cornea, normally functioning to maintain a moist and lubricated corneal surface, is impaired when the eye suffers from a dry-eye condition. Dry eye gives rise to discomforts such an ocular dryness, burning, scratching and inflammation. A more serious consequence of dry eye is loss of visual acuity and can, if not corrected, result in permanent eye damage. Dry-eye disease acts to degrade the exposed ocular surface and can cause a breakdown of corneal tissues. In an extreme case, this degradation of corneal tissue may necessitate a corneal transplant.

Dry eye may be exacerbated upon wearing contact lenses. The contact lens results in a menisci formation that promotes evaporation of natural moisture even when the eye has an adequate natural tear film. The usual prescribed treatment for dry eye is by topical application of a tear film substitute that adds a substantial volume of liquid to the surface of the eye. A typical composition functioning as a tear film substitute includes soluble polymer solutions. U.S. Pat. No. 4,421,740 discloses an artificial tear composition formed by an aqueous hypotonic solution of lecithin, a phospholipid, and a viscosity-adjusting agent.

There is a need for the improved treatment of inflammation appearing in diseases such as skin cancer, psoriasis, psoriatic arthritis, eczema, actinic keratosis, herpes, osteoarthritis, rheumatoid arthritis, and in eye conditions such as dry eye.

### SUMMARY OF THE INVENTION

Embodiments of the present invention relate to treatment of a skin disorder, a disorder associated with pain, fever, or inflammation, a proliferative disorder, or an ocular disorder. Aspects of the invention include topically administering a pharmaceutically acceptable formulation containing a therapeutically effective amount of a 4-phenylaminoquinoline compound, as well as methods of administration.

Accordingly, in a first aspect, the invention features a method of treating a skin disorder. Embodiments involve topically administering a therapeutically effective amount of a pharmaceutically acceptable formulation to a patient (for example, a human patient) in need thereof, where the pharmaceutically acceptable formulation includes an active compound having the formula:

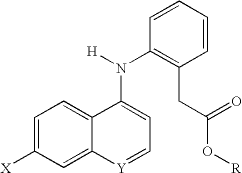

where

X is H, F, Cl, Br or CF₃;

Y is N, N-oxide (N—O), or a pharmaceutically acceptable acid addition salt thereof; and

R is H, lower alkyl, lower alkoxyalkyl, lower hydroxyalkyl, or a pharmaceutically acceptable cationic salt counterion.

In one embodiment, topically administering involves transdermal administration. The skin disorder may be a basal cell carcinoma, cutaneous metastatic breast cancer, pima squamous cell tumor, metastatic melanoma in the skin, malignancy or tumor in the skin, genital wart, corn on a foot, actinic keratosis, psoriasis, psoriatic arthritis, atopic dermatitis, liver spot, fungal lesion, skin lesion, or hair loss during pregnancy.

In a second aspect, the invention features a method of treating a disorder associated with pain, fever, or inflammation. Embodiments involve topically administering a therapeutically effective amount of a pharmaceutically acceptable formulation to a patient (for example, a human patient) in need thereof, where the pharmaceutically acceptable formulation includes an active compound having the formula:

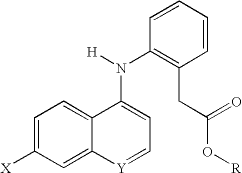

where

X is H, F, Cl, Br or CF₃;

Y is N, N-oxide (N—O), or a pharmaceutically acceptable acid addition salt thereof; and

R is H, lower alkyl, lower alkoxyalkyl, lower hydroxyalkyl, or a pharmaceutically acceptable cationic salt counterion.

The disorder associated with pain, fever, or inflammation may be rheumatic fever, lower back or neck pain, dysmenorrhea, a sprain or strain, headache, toothache, myositis, neuralgia, synovitis, arthritis (e.g., rheumatoid arthritis), a degenerative joint disease, osteoarthritis, gout and ankylosing spondylitis, bursitis, burns, or post-surgical or dental procedures, or pain and inflammation associated with herpes vesicles.

In a third aspect, the invention features a method of treating a proliferative disorder. Embodiments involve topically administering a therapeutically effective amount of a pharmaceutically acceptable formulation to a patient (for example, a human patient) in need thereof, where the pharmaceutically acceptable formulation includes an active compound having the formula:

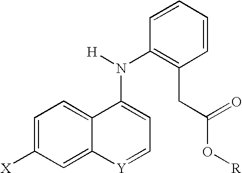

where

X is H, F, Cl, Br or CF₃;

Y is N, N-oxide (N—O), or a pharmaceutically acceptable acid addition salt thereof; and

R is H, lower alkyl, lower alkoxyalkyl, lower hydroxyalkyl, or a pharmaceutically acceptable cationic salt counterion.

The proliferative disorder may be, for example, diabetic retinopathy or includes tumor angiogenesis. Topical administration may involve transdermal or ocular administration.

In a fourth aspect, the invention features a method of treating an ocular disorder. Embodiments involve topically administering a therapeutically effective amount of a pharmaceutically acceptable formulation to a patient (for example, a human patient) in need thereof, where the pharmaceutically acceptable formulation includes an active compound having the formula:

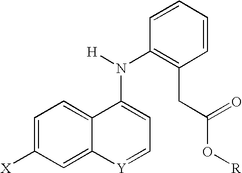

where

X is H, F, Cl, Br or CF₃;

Y is N, N-oxide (N—O), or a pharmaceutically acceptable acid addition salt thereof; and

R is H, lower alkyl, lower alkoxyalkyl, lower hydroxyalkyl, or a pharmaceutically acceptable cationic salt counterion.

The ocular disorder may be dry eye, non-infectious keratoconjunctivitis, superior limbic keratoconjunctivitis, toxic conjunctivitis, ocular cicatricial pemphigoid, Thygeson's superficial punctate keratopathy, uveitis, episcleritis, scleritis, iritis, blepharitis, keratitis, endophthalmitis, canaliculitis, dacryocystitis, preseptal cellulitis, orbital cellulitis. seborrheic blepharitis, meibomian gland dysfunction, acne rosacea, filamentary keratopathy, neurotrophic keratopathy, a corneal erosion, corneal dystrophy, iridocorneal endothelial syndrome, noninfectious ulcerative keratitis, a degeneration or corneal ectactic disorder, entropion, ectropion, trichiasis, lagophthalmos, or floppy eyelid syndrome. The non-infectious keratoconjunctivitis may be seasonal allergic conjunctivitis, atopic keratoconjunctivitis, vernal keratoconjunctivitis, contact dermatoconjunctivitis, giant papillary conjunctivitis, or contact lens-induced keratoconjunctivitis. The degeneration or corneal ectactic disorder may be ptyrygium, pinguecula, band-shaped keratopathy, Salzmann's nodular degeneration, keratoconus, or Terrien's marginal degeneration. In some instances, the ocular disorder may be due to ocular trauma and the ocular trauma may be a corneal abrasion, a corneal foreign body, a corneal laceration, or a chemical bum. In other instances, the ocular disorder may be an iatrogenic inflammatory condition related to post-laser assisted in situ keratomileusis (LASIK), post-laser epithelial keratomileusis (LASEK), post-photorefractive keratectomy (PRK), post-cataract surgery, or post-glaucoma filtration surgery.

The ocular disorder may also be infectious conjunctivitis, infectious keratitis, infectious endophthalmitis, and staphyloccocal blepharitis. The infectious conjunctivitis may be bacterial conjunctivitis, viral conjunctivitis, chlamydial conjunctivitis, or fungal conjunctivitis and the infectious keratitis may be bacterial keratitis, fungal keratitis, or acanthamoeba keratitis. Embodiments of the invention may further include the administration of an antimicrobial or antiviral agent.

The ocular disorder may also be an ophthalmic inflammatory condition such as conjunctivitis, iritis, uveitis, episcleritis, scleritis, keratitis, endophthalmitis, blepharitis, or an iatrogenic inflammatory condition. For example, the conjunctivitis may be non-infectious keratoconjunctivitis or infectious conjunctivitis.

In a fifth aspect, the invention features a pharmaceutically acceptable formulation suited to topical administration and containing a therapeutically effective amount of an active compound having the formula:

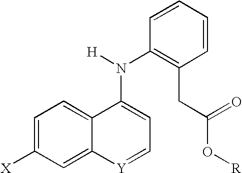

where

X is H, F, Cl, Br or CF₃;

Y is N, N-oxide (N—O), or a pharmaceutically acceptable acid addition salt thereof; and

R is H, lower alkyl, lower alkoxyalkyl, lower hydroxyalkyl, or a pharmaceutically acceptable cationic salt counterion.

In a sixth aspect, the invention features a kit including a pharmaceutically acceptable formulation containing an active compound having the formula:

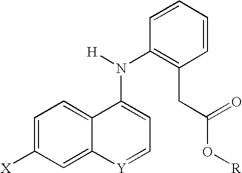

where

X is H, F, Cl, Br or CF₃;

Y is N, N-oxide (N—O), or a pharmaceutically acceptable acid addition salt thereof; and

R is H, lower alkyl, lower alkoxyalkyl, lower hydroxyalkyl, or a pharmaceutically acceptable cationic salt counterion;

and, typically, instructions for the topical administration of the pharmaceutically acceptable formulation.

In one embodiment of any of the foregoing aspects of the invention, the active compound is [2-(7-Trifluoromethyl-quinolin-4-ylamino)-phenyl]-acetic acid, or a pharmaceutically acceptable acid addition salt or cationic salt counterion thereof.

In another embodiment of any of the foregoing aspects of the invention, the active compound is [2-(7-Trifluoromethyl-quinolin-4-ylamino)-phenyl]-acetic acid methyl ester or a pharmaceutically acceptable acid addition salt thereof.

In a further embodiment, the active compound is soluble in a component of the pharmaceutically acceptable formulation. Other features and advantages of the invention will be apparent from the following Detailed Description, the drawings, and the claims.

### DETAILED DESCRIPTION

In general, the present invention relates to methods, by topical administration of an active compound, to treat any of a variety of disorders including skin disorders, disorders associated with pain, fever, or inflammation, proliferative disorders, or ocular disorders. Exemplary disorders include actinic keratosis, psoriatic arthritis, psoriasis, eczema, pain and/or inflammation associated with herpes vesicles, osteoarthritis, and rheumatoid arthritis, as well as inflammatory or proliferative disorders affecting the eye such as dry eye.

Non-steroidal anti-inflammatory drugs (NSAIDs) are drugs having analgesic, antipyretic, and anti-inflammatory effects, resulting in reduction of pain, fever, and inflammation. NSAIDs act by inhibiting cyclooxygenase enzymes, thereby reducing the conversion of arachidonic acid to prostaglandins. Most known NSAIDs act as non-selective inhibitors of cyclooxygenase by inhibiting both the cyclooxygenase-1 (COX-1) and cyclooxygenase-2 (COX-2) isoenzymes. In various embodiments, compounds in accordance with the present invention also exhibit such nonselectivity with respect to COX-1 and COX-2.

Disease-modifying anti-rheumatic drugs (DMARDs) are slow-acting anti-rheumatic drugs that are prescribed in addition to NSAIDs. NSAIDs reduce the acute and day-to-day inflammation while DMARDs slow down the biological processes that drive the persistent inflammation. DMARDs are slow acting and can take from one to three months to have a noticeable benefit in slowing the progression of the inflammation. Chloroquine possesses DMARD properties.

Cyclooxygenase (COX; prostaglandin endoperoxide synthase, EC 1.14.99.1) catalyzes the conversion of arachidonic acid to form the cyclic endoperoxides PGG and PGH) and subsequent metabolite products including prostaglandins and thromboxanes. There are two isoforms of this enzyme, cyclooxygenase-1 and -2 (COX-1 and COX-2). COX-1 is constitutively expressed in most cells. In contrast, COX-2 is not normally present but may be induced by certain serum factors, cytokines, growth factors and endotoxin. Inhibitors of COX-1 exhibit antithrombic activity and may also cause gastric ulceration. Inhibitors of COX-2 exhibit anti-inflammatory activity and may inhibit some mitogenic actions.

Compounds can be tested for their ability to inhibit the COX-1 enzyme by the following procedure: Human platelets are used. Test compound and/or vehicle is incubated with cells (5×10⁷/ml) in modified HEPES buffer pH 7.4 for 15 minutes at 37° C. The reaction is initiated by addition of 100 μM arachidonic acid for another 15 minute incubation period and terminated by further addition of 1 N HCl. An aliquot is removed and the amount of PGE 2 formed is determined spectrophotometrically by enzyme immunoassay (“EIA”) kit.

[2-(7-Trifluoromethyl-quinolin-4-ylamino)-phenyl]-acetic acid hydrochloride was screened at 6 concentrations to test for inhibition of the enzyme: 0.1 μM, 0.3 μM, 1 μM, 3 μM, 10 μM and 30 μM. [2-(7-Trifluoromethyl-quinolin-4-ylamino)-phenyl]-acetic acid hydrochloride inhibited the COX-1 enzyme with an IC₅₀ of <0.1 μM in this assay. When [2-(7-Trifluoromethyl-quinolin-4-ylamino)-phenyl]-acetic acid hydrochloride was tested separately at 3 concentrations (0.01 μM, 0.03 μM and 0.1 μM), an IC₅₀ of 0.081 μM was indicated.

Compounds can be tested for their ability to inhibit the COX-2 enzyme by the following procedure: Human recombinant Cyclooxygenase-2 expressed in Sf21 cells (Sigma, C-0858) is used. Test compound and/or vehicle is preincubated with 0.11 U cyclooxygenase-2, 1 mM reduced GSH, 500 μM phenol and 1 μM hematin for 15 minutes at 37° C. in Tris-HCl buffer pH 7.7. The reaction is initiated by addition of 0.3 μM arachidonic acid and terminated after 5 minutes by further addition of 1 N HCl. Following centrifugation, substrate conversion to PGE 2 is measured by an Amersham EIA kit.

[2-(7-Trifluoromethyl-quinolin-4-ylamino)-phenyl]-acetic acid hydrochloride was screened at 7 concentrations to test for inhibition of the enzyme: 0.01 uM, 0.03 μM, 0.1 μM, 0.3 μM, 1 μM, 3 μM and 10 μM. [2-(7-Trifluoromethyl-quinolin-4-ylamino)-phenyl]-acetic acid hydrochloride inhibited the COX-2 enzyme with an IC₅₀ of 0.061 μM in this assay.

A compound can be tested for topical anti-inflammatory activity by measuring the degree of inhibition of edema produced in a mouse ear that has been irritated by the application of a solution of a phorbol ester. [2-(7-Trifluoromethyl-quinolin-4-ylamino)-phenyl]-acetic acid sodium salt was tested for its anti-inflammatory activity as follows: Groups of 6 CD-1 (Crl.) derived male mice weighing 24±2 g were used. Phorbol 12-Myristate 13-Acetate (PMA 4 μg in 20 μl of Acetone) was applied topically to the anterior and posterior surfaces of the right ear to each animal. Vehicle (Ethanol:Acetone/1:1, 20 μl/ear) and [2-(7-Trifluoromethyl-quinolin-4-ylamino)-phenyl]-acetic acid sodium salt at 3 mg/ear were each applied 30 minutes before and 15 minutes after Phorbol 12-Myristate 13-Acetate challenge. Indomethacin (3 mg in 20 μl/ear of acetone:ethanol/1:1) as the positive control was similarly applied at the same timing. Ear swelling was then measured by a Dyer model micrometer gauge at 6 hours after Phorbol 12 Myristate 13-Acetate application as an index of inflammation. Percent inhibition was calculated according to the formula: ([Ic−It]/Ic)×100%, where Ic and It refer to increase of ear thickness (mm) in control and treated mice, respectively. Inhibition of 30 percent or more (≧30%) in ear swelling is considered significant anti-inflammatory activity.

[2-(7-Trifluoromethyl-quinolin-4-ylamino)-phenyl]-acetic acid sodium salt caused significant inhibition (80%) of the PMA-induced ear swelling vs vehicle control. When similarly tested, [2-(7-Trifluoromethyl-quinolin-4-ylamino)-phenyl]-acetic acid hydrochloride also caused significant inhibition (75%). Concurrently, Indomethacin (3 mg/ear×2) caused a significant decrease (77% inhibition) in the ear swelling relative to the vehicle-treated control. When [2-(7-Trifluoromethyl-quinolin-4-ylamino)-phenyl]-acetic acid methyl ester was tested in this assay, the inhibition of ear swelling was not significant (20% inhibition).

The active compounds described herein are NSAIDs that also possess DMARD properties and are represented by the formula:

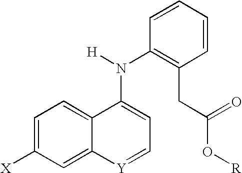

where X is H, F, Cl, Br or CF₃; Y is N or N-oxide; and R is H, lower alkyl, lower alkoxyalkyl, or lower hydroxyalkyl. The compound may be a pharmaceutically acceptable acid addition salt, a pharmaceutically acceptable cationic salt counterion, a non-charged molecule or zwitterionic. The active compound may be [2-(7-Trifluoromethyl-quinolin-4-ylamino)-phenyl]-acetic acid, a pharmaceutically acceptable cationic salt counterion thereof, a pharmaceutically acceptable acid addition salt thereof or [2-(7-Trifluoromethyl-quinolin-4-ylamino)-phenyl]-acetic acid methyl ester or a pharmaceutically acceptable acid addition salt thereof. Other compounds useful in accordance herewith include [2-(7-Chloro-quinolin-4-ylamino)-phenyl]-acetic acid, a pharmaceutically acceptable cationic salt counterion thereof, a pharmaceutically acceptable acid addition salt thereof or [2-(7-Chloro-quinolin-4-ylamino)-phenyl]-acetic acid methyl ester, a pharmaceutically acceptable acid addition salt thereof.

### 1. Definitions

An “active compound” as used herein refers to a compound having the formula:

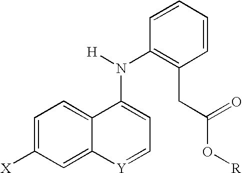

where X is H, F, Cl, Br or CF₃; Y is N or N-oxide; and R is H, lower alkyl, lower alkoxyalkyl, or lower hydroxyalkyl. The compound may be a pharmaceutically acceptable acid addition salt, a pharmaceutically acceptable cationic salt counterion, a non-charged molecule or zwitterionic. In some embodiments, the active compound is [2-(7-Trifluoromethyl-quinolin-4-ylamino)-phenyl]-acetic acid, a pharmaceutically acceptable cationic salt counterion thereof, a pharmaceutically acceptable acid addition salt thereof or [2-(7-Trifluoromethyl-quinolin-4-ylamino)-phenyl]-acetic acid methyl ester or a pharmaceutically acceptable acid addition salt thereof. Other compounds suitable for use in the present invention include [2-(7-Chloro-quinolin-4-ylamino)-phenyl]-acetic acid, a pharmaceutically acceptable cationic salt counterion thereof, a pharmaceutically acceptable acid addition salt thereof or [2-(7-Chloro-quinolin-4-ylamino)-phenyl]-acetic acid methyl ester, a pharmaceutically acceptable acid addition salt thereof or a combination of such active compounds.

The term “skin disorder” as used herein refers broadly to a disease or abnormal condition affecting the skin. Exemplary skin disorders that can be treated by active compounds described herein include, but are not limited to, basal cell carcinoma, cutaneous metastatic breast cancer, pima squamous cell tumor, metastatic melanoma in the skin, malignancies and tumors in the skin, genital warts, corns on the feet, actinic keratosis, psoriasis, psoriatic arthritis, hair loss during pregnancy, atopic dermatitis, liver spots, fungal lesions, or skin lesions.

The term “ocular disorder” as used herein refers broadly to a disease or abnormal condition affecting the eye. Exemplary ocular disorders that can be treated by active compounds described herein include, but are not limited to, dry eye, non-infectious keratoconjunctivitis, superior limbic keratoconjunctivitis, toxic conjunctivitis, ocular cicatricial pemphigoid, Thygeson's superficial punctate keratopathy, uveitis, episcleritis, scleritis, iritis, blepharitis, keratitis, endophthalmitis, canaliculitis, dacryocystitis, preseptal cellulitis, orbital cellulitis, seborrheic blepharitis, meibomian gland dysfunction, acne rosacea, filamentary keratopathy, neurotrophic keratopathy, corneal erosions, corneal dystrophies, iridocorneal endothelial syndrome, noninfectious ulcerative keratitis, a degeneration or corneal ectactic disorder, entropion, ectropion, trichiasis, lagophthalmos, floppy eyelid syndrome, seasonal allergic conjunctivitis, atopic keratoconjunctivitis, vernal keratoconjunctivitis, contact dermatoconjunctivitis, giant papillary conjunctivitis, or contact lens-induced keratoconjunctivitis, ptyrygium, pinguecula, band-shaped keratopathy, Salzmann's nodular degeneration, keratoconus, and Terrien's marginal degeneration, infectious conjunctivitis, infectious keratitis, infectious endophthalmitis, and staphyloccocal blepharitis, bacterial conjunctivitis, viral conjunctivitis, chlamydial conjunctivitis, fungal conjunctivitis, bacterial keratitis, fungal keratitis, acanthamoeba keratitis, or infectious conjunctivitis.

The term “dry eye” as used herein refers to a condition wherein the relative amount of lachrymal fluid is below average (typically by one standard deviation) or abnormal in quality. Dry eye can occur in the presence or absence of corneal and/or conjunctival lesions (Yamada, M. et al., Folia Ophthalmol. Jpn., 43:1289-1293 (1992)). Examples of dry eye include dry eye observed in hypolacrimation, alacrima, xerophthalmia, Sjogren syndrome, keratoconjunctivitis sicca, Stevens-Johnson syndrome, ocular pemphigoid, marginal blepharitis, diabetes and the like, dry eye observed after cataract operation, dry eye in conjunction with allergic conjunctivitis and the like, and dry eye due to hypolacrimation caused by increased visual display terminal (VDT) work, or to an arid environment.

The term “ocular trauma” as used herein broadly refers to an injury to the eye. Exemplary ocular traumas that can be treated by active compounds described herein include, but are not limited to, corneal abrasion, a corneal foreign body, a corneal laceration, or a chemical burn.

The term “herpes vesicles” as used herein refers to small fluid-filled blisters on the skin ranging in size from a pinpoint to 5 or 10 millimeters in diameter that manifest during an outbreak associated with HSV1 or HSV2.

The term “iatrogenic inflammatory condition” as used herein refers to an inflammatory process or response due to a medical treatment or intervention. Exemplary iatrogenic inflammatory conditions that can be treated as described herein include, but are not limited to, post-laser-assisted in situ keratomileusis (LASIK), post-laser epithelial keratomileusis (LASEK), post-photorefractive keratectomy (PRK), post-cataract surgery, or post-glaucoma filtration surgery.

The term “antimicrobial agent” as used herein refers to an agent that inhibits the growth of microorganisms. Exemplary microorganisms are bacteria and fungi.

The term “antiviral agent” as used herein refers to an agent that destroys or weakens a virus or interferes with its ability to replicate.

The term “proliferative disorder” as used herein refers to a condition characterized by uncontrolled, abnormal or excessive growth of cells. Exemplary proliferative disorders that can be treated by active compounds described herein include, but are not limited to, diabetic retinopathy and tumor angiogenesis.

The term “inflammation,” “inflammatory disorder” or “inflammatory condition” as used herein refers to activation of the immune system to an abnormal level that leads to disease or a protective reaction by tissues resulting from injury, irritation, damage or destruction. Acute inflammation is characterized by pain, heat, redness, swelling and loss of tissue function. Exemplary inflammatory disorders treatable as described herein include, but are not limited to, rheumatic fever, lower back or neck pain, dysmenorrhea, sprains and strains, headache, toothache, myositis, neuralgia, synovitis, arthritis, rheumatoid arthritis, degenerative joint diseases, osteoarthritis, gout and ankylosing spondylitis, bursitis, bums, post-surgical or dental procedures, and inflammation associated with herpes vesicles.

The term “pharmaceutically acceptable formulation” as used herein refers to a composition including a pharmaceutically acceptable carrier and an active compound.

A “pharmaceutically acceptable carrier” as used herein refers to a vehicle capable of suspending or dissolving the active compound, and having the properties of being substantially nontoxic and non-inflammatory in a patient. A pharmaceutically acceptable carrier may be a liquid or cream and, desirably, is suitable for topical application, e.g., application to the skin. As such, the term “pharmaceutically acceptable carrier” encompasses carrier materials approved for use in topical cosmetics. Moreover, a pharmaceutically acceptable carrier may include a pharmaceutically acceptable additive, such as a preservative, antioxidant, fragrance, emulsifier, dye, or excipient known or used in the field of drug formulation and that does not significantly interfere with the therapeutic effectiveness of the biological activity of the active agent, and that is non-toxic to the patient.

The term “excipient” is used herein to describe any ingredient other than an active compound described herein.

As used herein, the term “pharmaceutical patch” refers to a pad containing an embedded active compound to be placed on the exterior surface of a patient for absorption of the active compound into the bloodstream, skin or underlying tissue. The patch is typically placed on the skin and the compound is released gradually from the patch over time. The patch may be an adhesive patch.

As used herein, the term “therapeutically effective amount” refers to an amount of an active compound that, when administered to a patient, reduces, eliminates or prevents a skin disorder, a disorder associated with pain, fever, or inflammation, a proliferative disorder, or an ocular disorder. A therapeutically effective amount of a pharmaceutical formulation may contain the active compound in a concentration range of about 0.000001 to 10% weight/volume (“% w/v”). In various embodiments, the compound is [2-(7-Trifluoromethyl-quinolin-4-ylamino)-phenyl]-acetic acid, [2-(7-Trifluoromethyl-quinolin-4-ylamino)-phenyl]-acetic acid methyl ester, [2-(7-Chloro-quinolin-4-ylamino)-phenyl]-acetic acid or [2-(7-Chloro-quinolin-4-ylamino)-phenyl]-acetic acid methyl ester or when applicable, pharmaceutically acceptable cationic salt counterions thereof or pharmaceutically acceptable acid addition salts thereof.

“Topical administration” or “topically administering” as used herein refers to the application of a pharmaceutically acceptable formulation to the external surface of a patient, such that the active compound enters the underlying tissue. The external surface may be the skin and topical administration may involve application of a pharmaceutically acceptable formulation to intact, broken, or raw skin or to an open skin wound.

“Transdermal administration” or “transdermally administering” as used herein refers to the diffusion of an agent across the barrier of the skin resulting from topical administration or other application of a pharmaceutically acceptable formulation.

“Ocular administration” as used herein refers to administration of a pharmaceutically acceptable formulation topically to the eye.

A “pharmaceutically acceptable acid addition salt” is derived from a basic active compound and an organic acid or an inorganic acid. Exemplary pharmaceutically acceptable acid addition salts derived from organic acids include acetate, adipate, alginate, ascorbate, aspartate, benzenesulfonate, benzoate, butyrate, camphorate, camphersulfonate, citrate, cyclopentanepropionate, digluconate, dodecylsulfate, ethanesulfonate, fumarate, glucoheptonate, glycerophosphate, heptonate, hexanoate, 2-hydroxy-ethanesulfonate, isethionate, lactobionate, lactate, laurate, lauryl sulfate, malate, maleate, malonate, methanesulfonate, 2-naphthalenesulfonate, nicotinate, oleate, oxalate, palmitate, pamoate, pectinate, persulfate, 3-phenylpropionate, picrate, pivalate, propionate, stearate, succinate, tartrate, thiocyanate, toluenesulfonate, undecanoate, and valerate salts, and the like. Exemplary pharmaceutically acceptable acid addition salts derived from inorganic acids include bisulfate, sulfate, borate, hydrobromide, hydrochloride, hydroiodide, hemisulfate, nitrate, phosphate salts and the like. Particular embodiments involve hydrochloride, hydrobromide, methanesulfonate, sulfate, hemisulfate or bisulfate.

A “pharmaceutically acceptable cationic salt counterion” refers to a positively charged molecule or atom that is balanced by a negatively charged active compound carboxylate anion. Exemplary pharmaceutically acceptable cationic salt counterions include metal salts such as, for example, aluminum, calcium, copper, ferric, ferrous, lithium, magnesium, manganic salts, manganous, potassium, sodium, zinc, and the like as well as nontoxic ammonium, quarternary ammonium and amine cations including, but not limited to ammonium, tetramethylammonium, tetraethylammonium, methylamine, dimethylamine, trimethylamine, triethylamine, ethylamine, naturally occurring substituted amines, cyclic amines, arginine, betnine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, procaine, purines, theobromine, tripropylamine, tromethamine, triethanolamine and the like.

The term “lower alkyl” refers to C₁-C₇ alkyl groups that have a straight, branched or cyclic configuration. Examples of lower alkyl groups include methyl, ethyl, propyl, isopropyl, butyl, s-butyl, t-butyl, pentyl, hexyl, heptyl, cyclopropyl, cyclobutylmethyl, cycloheptyl, and the like. In some embodiments, a lower alkyl is methyl or ethyl.

The term “lower alkoxyalkyl” refers to ethoxyethyl, methoxyethyl, methoxypropyl groups, and the like.

The term “lower hydroxyalkyl” refers to lower alkyl groups that are further substituted with 1 or 2 hydroxyl groups. Exemplary lower hydroxyalkyl groups include 2-hydroxyethyl, 2-hydroxypropyl, 2,3-dihydroxypropyl, and the like. In various embodiments, the hydroxyalkyl group is 2,3-dihydroxypropyl.

### 2. Pharmaceutical Formulations

2.1 Topical Formulations

The present invention includes methods of treating inflammatory and proliferative disorders including, but not limited to, rheumatic fever, lower back or neck pain, dysmenorrhea, sprains and strains, headache, toothache, myositis, neuralgia, synovitis, arthritis, rheumatoid arthritis, degenerative joint diseases, osteoarthritis, gout and ankylosing spondylitis, bursitis, bums, post-surgical or dental procedures, diabetic retinopathy, tumor angiogenesis, basal cell carcinoma, cutaneous metastatic breast cancer, pima squamous cell tumor, metastatic melanoma in the skin, malignancies and tumors in the skin, genital warts, corns on the feet, actinic keratosis, psoriasis, psoriatic arthritis, hair loss during pregnancy, atopic dermatitis, liver spots, fungal lesions, skin lesions, and pain or inflammation associated with herpes vesicles using topical pharmaceutically acceptable formulations containing an active compound having the formula:

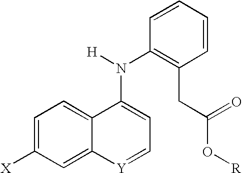

where X is H, F, Cl or CF₃; Y is N or N-oxide; and R is H, lower alkyl, lower alkoxyalkyl, or lower hydroxyalkyl. The compound may be a pharmaceutically acceptable acid addition salt, a pharmaceutically acceptable cationic salt counterion, a non-charged molecule or zwitterionic. In some embodiments, the active compound is [2-(7-Trifluoromethyl-quinolin-4-ylamino)-phenyl]-acetic acid, a pharmaceutically acceptable cationic salt counterion thereof, a pharmaceutically acceptable acid addition salt thereof or [2-(7-Trifluoromethyl-quinolin-4-ylamino)-phenyl]-acetic acid methyl ester or a pharmaceutically acceptable acid addition salt thereof. Other useful compounds for use in the present invention include [2-(7-Chloro-quinolin-4-ylamino)-phenyl]-acetic acid, a pharmaceutically acceptable cationic salt counterion thereof, a pharmaceutically acceptable acid addition salt thereof or [2-(7-Chloro-quinolin-4-ylamino)-phenyl]-acetic acid methyl ester or a pharmaceutically acceptable acid addition salt.

Pharmaceutically acceptable formulations for use in accordance with the present invention thus can be formulated in conventional manner using one or more physiologically acceptable carriers comprising excipients and auxiliaries that facilitate processing of the active compounds into preparations that can be used medically or topically. Proper formulation is dependent upon the desired product chosen. Non-limiting exemplary formulations are provided below.

The topical formulations useful in the subject invention can be made into a wide variety of product types. These include, but are not limited to, lotions, creams, gels, sticks, sprays, ointments, pastes, mousses, and cosmetics. The product types can include several types of carrier systems including, but not limited to solutions, emulsions, gels, solids, and liposomes. Techniques for formulation and administration are standard in the art and can be found, for example, in Remington: The Science and Practice of Pharmacy, 20^{th} edition,” Lippincott Williams & Wilkins, Philadelphia, Pa. Eds Gennaro A. R. et al, 2000 (hereafter “Remington,” the entire disclosure of which is hereby incorporated by reference). The formulation can be selected to maximize delivery to a desired target site in the body such as the skin. As is appreciated by the ordinarily skilled artisan, the specific base to be used is one that provides for optimum delivery for the active agent chosen for a given formulation, and, preferably, provides for other desired characteristics as well, e.g., emolliency or the like.

Lotions, which are preparations that are to be applied to the skin surface, are typically liquid or semi-liquid. Lotions may be formulated with an aqueous or oily base and will in general also contain one or more emulsifying agents, stabilizing agents, dispersing agents, suspending agents, thickening agents, preservatives or coloring agents.

Creams containing the active agent for delivery according to the present invention are viscous liquid or semisolid emulsions, usually oil-in-water or water-in-oil. Cream bases generally contain an oil phase, an emulsifier and an aqueous phase. The oil phase usually, although not necessarily, contains ingredients such as mineral, animal and vegetable oils and waxes, fatty acids, alcohols, amides and esters; the aqueous phase usually, although not necessarily, exceeds the oil phase in volume, and generally contains ingredients such as alcohols, polyols, humectants, viscosity builders and preservatives. The emulsifier in a cream formulation, as described in Remington, is generally a nonionic, anionic, cationic or amphoteric surfactant.

Gel formulations can also be used in connection with the present invention. As is appreciated by those working in the field of topical drug formulation, gels are viscous semisolid systems. Gels contain organic macromolecules distributed substantially uniformly throughout the carrier liquid, which is typically aqueous, but also may contain solvents, co-solvents and other pharmaceutically known and acceptable adjuvants.

Ointments, which are semisolid preparations, are typically based on petrolatum or other petroleum derivatives. As with other carriers or vehicles, an ointment base should be inert, stable, nonirritating and non-sensitizing. As described, for example, in Remington at pages 845-849, ointment bases may be grouped in four classes: oleaginous bases; absorption bases; water-removable bases; and water-soluble bases. Oleaginous ointment bases include, for example, vegetable oils, fats obtained from animals, and semisolid hydrocarbons obtained from petroleum. Absorption bases, also known as emulsifiable ointment bases, contain little or no water and include, for example, hydroxystearin sulfate, anhydrous lanolin and hydrophilic petrolatum. Absorption bases are generally water-in-oil (W/O) emulsions and include, for example, cetyl alcohol, glyceryl monostearate, lanolin and stearic acid. Water-soluble ointment bases can be prepared from polyethylene glycols of varying molecular weight.

Useful formulations of the invention also encompass sprays. Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the drug or active agent can be dissolved. Upon delivery to the skin, the carrier evaporates, leaving concentrated active agent at the site of administration.

A topical pharmaceutical formulation for use in the present invention may also include suitable solid or gel phase carriers for modifying viscosity. Examples of such carriers include, but are not limited to, calcium carbonate, calcium phosphate, various sugars, starches, cellulose derivatives, gelatin, and polymers such as polyethylene glycols.

Further, a topical pharmaceutical formulation may include a suitable emulsifier, i.e., an agent that enhances or facilitates mixing and suspending oil-in-water or water-in-oil. An emulsifying agent for use in the invention may consist of a single emulsifying agent or may be a blend of emulsifying agents and may be a nonionic, anionic or cationic surfactant or a blend of two or more such surfactants. Such surface-active agents are described, for example, in “McCutcheon's Detergent and Emulsifiers,” North American Edition, 1980 Annual published by the McCutcheon Division, MC Publishing Company, 175 Rock Road, Glen Rock, N.J. 07452, USA.

Especially suitable nonionic emulsifying agents for inclusion in the pharmaceutically acceptable formulations for use in the present invention are those with a hydrophile-lipophile balance (HLB) as determined by the method described, for example, by Paul L. Lindner in “Emulsions and Emulsion”, edited by Kenneth Lissant, published by Dekker, New York, N.Y., 1974, pages 188-190. Examples of such nonionic emulsifiers include, but are not limited to, “BRIJ 72,” the trade name for a polyoxyethylene (2) stearyl ether having an HLB of 4.9; “BRIJ 721,” the trade name for a polyoxyethylene (21) stearyl ether having an HLB of 15.5.

A topical pharmaceutical formulation may also contain suitable emollients. Emollients are materials that may be used for the prevention or relief of dryness, as well as for the protection of the skin. Useful emollients include, but are not limited to, cetyl alcohol, isopropyl myristate, stearyl alcohol, and the like. A wide variety of suitable emollients are known in the art and can be used in the formulations encompassed by the invention. See e.g., Sagarin, Cosmetics, Science and Technology, 2nd Edition, Vol. 1, pp. 32-43 (1972), and U.S. Pat. No. 4,919,934, to Deckner et al., both of which are incorporated herein by reference in their entirety.

A topical pharmaceutical formulation for use in the methods of the invention may also include suitable antioxidants, i.e., substances that inhibit oxidation. Antioxidants suitable for use in accordance with the present invention include, but are not limited to, butylated hydroxytoluene, ascorbic acid, sodium ascorbate, calcium ascorbate, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone and tocopherols such as vitamin E, and the like, including pharmaceutically acceptable salts and esters of these compounds. Moreover, topical pharmaceutical formulations for use in the present invention may also include suitable preservatives. Preservatives are compounds added to a pharmaceutical formulation to act as an anti-microbial agent. Among preservatives known in the art as being effective and acceptable in parenteral formulations are benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. See, e.g., Wallhausser, K.-H., Develop. Biol. Standard, 24:9-28 (1974) (S. Krager, Basel).

A topical pharmaceutical formulation for use in the present invention may further contain suitable chelating agents to form complexes with metal cations which do not cross a lipid bilayer. Examples of suitable chelating agents include ethylene diamine tetraacetic acid (EDTA), ethylene glycol-bis(beta-aminoethyl ether)-N,N,N′,N′-tetraacetic acid (EGTA) and 8-Amino-2-[(2-amino-5-methylphenoxy)methyl]-6-methoxyquinoline-N,N,N′,N′-tetraacetic acid, tetrapotassium salt (QUIN-2).

Topical pharmaceutical formulations useful for the methods of the invention may also include suitable neutralizing or buffering agents used to adjust the pH of the formulation to optimize the delivery of the drug.

Further, a topical pharmaceutical formulation may include suitable hydrophilic gelling agents. These components are, for example, diffusible compounds capable of increasing the viscosity of a polymer-containing solution through the interaction of the agent with the polymer. Also useful herein are hydrophilic gelling agents such as the acrylic acid/ethyl acrylate copolymers and the carboxyvinyl polymers sold by the B. F. Goodrich Company under the trademark of CARBOPOL resins. These resins contain a colloidally water-soluble polyalkenyl-polyether-crosslinked polymer of acrylic acid, crosslinked with from 0.75% to 2.00% of a crosslinking agent such as polyallyl sucrose or polyallyl pentaerythritol. A useful viscosity increasing agent is, for example, CARBOPOL Ultrez 10.

A topical pharmaceutical formulation may also contain one or more suitable solvents and cosolvents. Suitable solvents and cosolvents which are exemplified may include ingredients such as ethanol, propylene glycol, glycerin, dipropylene glycol and polyethylene glycol. In addition, a topical pharmaceutical formulation for use in the methods of the invention may include one or more suitable skin penetration enhancers. Suitable excipients are known in the art to be skin penetration enhancers (as described, for example, in Osborne D. W. and Henke J. J., “Skin penetration enhancers cited in the technical literature” Pharm. Tech. 21:58-66, 1997). Examples of skin penetration enhancers include water, ethanol, propylene glycol, oleic acid, oleyl alcohol, sodium lauryl sulfate, dimethylsulfoxide, 1-dodecylazacycloheptan-2-one (trade name AZONE), N-methyl-2-pyrolidinone, 2-pyrolidinone, D-limonene, 1,8-cineole, urea, and menthol are just a few of the known penetration enhancers. Diethylene glycol monoethyl ether NF (CAS number 111-90-0, INCI name ethoxydiglycol, trade name TRANSCUTOL) (see, for example, Watkinson A. C. et al., “Aspects of the transdermal delivery of prostaglandins”, Int. J. Pharm. 74:229-236, 1991; Rojas J. et al., “Optimization of binary and ternary solvent systems in the percutaneous absorption of morphine base”, STP Pharma Sciences, 1:70-75, 1991; Watkinson A. C., Ph.D. Thesis, University of Wales, 1991; Ritschel W. A. et al., “Development of an intracutaneous depot for drugs. Binding, drug accumulation and retention studies”, Skin Pharmacol. 4:235-245, 1991).

Diethylene glycol monoethyl ether NF (DGME) is a useful solvent for many drugs, especially non-lipophilic drugs having very low water solubility. In vitro skin absorption studies have shown increased flux values for compounds dissolved in DGME; however, DGME does not fluidize the stratum corneum lipids (Harrison J. E. et al., “The relative effect of Azone and Transcutol on permeant diffusivity and solubility in human stratum corneum,” Pharm. Res., 13:542-546, 1996), nor does DGME decrease the lag time associated with the permeant (Rojas J. et al., “Optimization of binary and ternary solvent systems in the percutaneous absorption of morphine base”, STP Pharma Sciences, 1:70-75, 1991). These additional penetration-enhancing compounds can be used when desired in the pharmaceutical compositions described herein in the conventional range of from about 0.1 to about 10% and preferably about 1.0% to about 5.0% by weight of the topical composition.

Liquid forms, such as lotions suitable for topical administration or suitable for cosmetic application, may include a suitable aqueous or nonaqueous vehicle with buffers, suspending and dispensing agents, thickeners, penetration enhancers, and the like. Solid and semi-solid forms such as creams, ointments, sticks or pastes or the like may include, for example, any of the following ingredients, water, oil, alcohol or grease as a substrate with surfactant, polymers such as polyethylene glycol, thickeners, solids and the like. Liquid or solid formulations may include enhanced delivery technologies such as liposomes, microsomes, microsponges, patches, and the like.

### 3. Topical Administration

The active compounds for use in the methods described herein can be administered in a pharmaceutically acceptable topical (e.g., transdermal) formulation. Topical treatment regimens according to the practice of the invention may include applying the composition directly to the skin at the application site, from one to several times daily. The present invention includes kits containing the active compound in a pharmaceutically acceptable formulation and instructions for topical administration. Topical administration includes delivery methods in the form of pharmaceutical patches.

A pharmaceutically acceptable topical formulation may include a pharmaceutically acceptable carrier such as water, oils (including vegetable and mineral oils), cream bases, lotion bases, ointment bases, and the like. These bases include suspending agents, thickeners, penetration enhancers, and the like. Topical and transdermal formulations are well known to those in the art of cosmetics and topical pharmaceuticals and are described, for example, in Chapter 44 of Remington.

Topical (e.g., transdermal) formulations may also include pharmaceutically acceptable vehicles. Additives for topical formulations are well-known in the art, and may be added to the topical composition, as long as they are pharmaceutically acceptable and not deleterious to the epithelial cells or their function. Further, the additives should not cause deterioration in the stability of the formulation, in particular, of the active compound. For example, inert fillers, anti-irritants, tackifiers, excipients, fragrances, opacifiers, antioxidants, gelling agents, stabilizers, surfactants, emollients, coloring agents, preservatives, buffering agents, other permeation enhancers, and other conventional components of transdermal delivery devices as are known in the art. Excipients generally are carriers, diluents and/or vehicles used in formulating drug compositions. Excipients are standard in the art and examples of excipients and their application can be found, for instance, in Katz, M., Drug Design 4:93-148, 1973.

Penetration or permeation through the skin of an active compound may be enhanced by an agent (e.g., p20 solvents) or a mixture of agents which, alone or in combination, act to increase the permeability of the skin to a drug. The enhanced permeation effected through the use of such enhancers can be observed, for example, by measuring the rate of diffusion of the drug through animal or human skin using a diffusion cell apparatus. A diffusion cell is described by Merritt et al. “Diffusion Apparatus for Skin Penetration,” J. of Controlled Release, 1:161-162, 1984. Topical administration of a pharmaceutical agent can result in a limited distribution of the agent to the skin and surrounding tissues or, when the agent is removed from the treatment area by the bloodstream, can result in systemic distribution of the agent. However, transdermal administration desirably results in the diffusion of an agent across the barrier of the skin resulting from topical administration or other application of a pharmaceutically acceptable formulation. Transdermal delivery includes injection or other delivery through any portion of the skin or mucous membrane and absorption or permeation through the remaining portion. Absorption through intact skin can be enhanced by placing the active agent in an appropriate pharmaceutically acceptable vehicle before application to the skin. Passive topical administration may consist of applying the active agent directly to the treatment site in combination with emollients or penetration enhancers.

A topically (e.g., transdermally) administrable pharmaceutical formulation may also include an amount of a form of hyaluronic acid sufficient to transport the composition through the skin of a patient into the epidermis or dermis where the composition remains until discharged via the lymphatic system. In various embodiments, the active compound is 1-5% by weight of the formulation and hyaluronic acid is 1-3% by weight of the formulation. Suitable forms of hyaluronic acid have a molecular weight greater than about 150,000 daltons and less than 750,000 daltons. Salts of hyaluronic acid are also useful in connection with the present invention.

### 4. Ocular Administration

The present invention also features methods of treating inflammatory and proliferative disorders that include, or result from, ocular disorders, ocular trauma and iatrogenic inflammatory conditions including, but not limited to, dry eye, non-infectious keratoconjunctivitis, superior limbic keratoconjunctivitis, toxic conjunctivitis, ocular cicatricial pemphigoid, Thygeson's superficial punctate keratopathy, uveitis, episcleritis, scleritis, iritis, blepharitis, keratitis, endophthalmitis, canaliculitis, dacryocystitis, preseptal cellulitis, orbital cellulitis. seborrheic blepharitis, meibomian gland dysfunction, acne rosacea, filamentary keratopathy, neurotrophic keratopathy, corneal erosions, corneal dystrophies, iridocorneal endothelial syndrome, noninfectious ulcerative keratitis, a degeneration or corneal ectactic disorder, entropion, ectropion, trichiasis, lagophthalmos, floppy eyelid syndrome, seasonal allergic conjunctivitis, atopic keratoconjunctivitis, vernal keratoconjunctivitis, contact dermatoconjunctivitis, giant papillary conjunctivitis, or contact lens-induced keratoconjunctivitis, ptyrygium, pinguecula, band-shaped keratopathy, Salzmann's nodular degeneration, keratoconus, and Terrien's marginal degeneration, infectious conjunctivitis, infectious keratitis, infectious endophthalmitis, and staphyloccocal blepharitis, bacterial conjunctivitis, viral conjunctivitis, chlamydial conjunctivitis, fungal conjunctivitis, bacterial keratitis, fungal keratitis, acanthamoeba keratitis, infectious conjunctivitis, corneal abrasion, a corneal foreign body, a corneal laceration, a chemical burn, post-laser assisted in situ keratomileusis (LASIK), post-laser epithelial keratomileusis (LASEK), post-photorefractive keratectomy (PRK), post-cataract surgery, or post-glaucoma filtration surgery using ocular administration of topical pharmaceutical compositions containing an active compound having the formula:

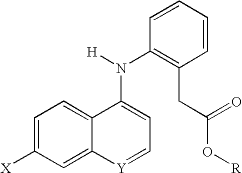

where X is H, F, Cl, Br, or CF₃; Y is N or N-oxide; and R is H, lower alkyl, lower alkoxyalkyl, or lower hydroxyalkyl. The compound may be a pharmaceutically acceptable acid addition salt, a pharmaceutically acceptable cationic salt counterion, a non-charged molecule or zwitterionic. In some embodiments, the active compound is [2-(7-Trifluoromethyl-quinolin-4-ylamino)-phenyl]-acetic acid, a pharmaceutically acceptable cationic salt counterion thereof, a pharmaceutically acceptable acid addition salt thereof or [2-(7-Trifluoromethyl-quinolin-4-ylamino)-phenyl]-acetic acid methyl ester or a pharmaceutically acceptable acid addition salt thereof. Other useful compounds for use in ocular administration include [2-(7-Chloro-quinolin-4-ylamino)-phenyl]-acetic acid, a pharmaceutically acceptable cationic salt counterion thereof, a pharmaceutically acceptable acid addition salt thereof or [2-(7-Chloro-quinolin-4-ylamino)-phenyl]-acetic acid methyl ester or a pharmaceutically acceptable acid addition salt.

The pharmaceutical formulations may be present in a kit containing instructions for ocular administration and may include pharmaceutical carriers known in the art such as, but are not limited to, ophthalmically acceptable solutions, suspensions and other dosage forms for topical administration. Aqueous solutions are generally preferred, based on ease of formulation, biological compatibility, as well as a patient's ability to easily administer such compositions, for example, by means of instilling one to two drops of the solutions in the affected eye. However, the compositions may also be suspensions, viscous or semi-viscous gels, or other types of solid or semi-solid compositions.

The pharmaceutical formulation for ocular administration may contain a therapeutically effective amount of active compound having the formula:

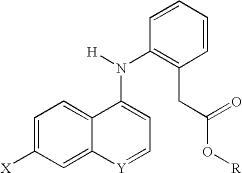

where X is H, F, Cl or CF₃; Y is N or N-oxide; and R is H, lower alkyl, lower alkoxyalkyl, or lower hydroxyalkyl. The compound may be a pharmaceutically acceptable acid addition salt, a pharmaceutically acceptable cationic salt counterion, a non-charged molecule or zwitterionic that, when administered to a patient, reduces, eliminates or prevents ophthalmic inflammation. For example, the active compound may be present in a concentration range of about 0.000001 to 10% weight/volume (“% w/v”). In some embodiments, the compound is [2-(7-Trifluoromethyl-quinolin-4-ylamino)-phenyl]-acetic acid, [2-(7-Trifluoromethyl-quinolin-4-ylamino)-phenyl]-acetic acid methyl ester, [2-(7-Chloro-quinolin-4-ylamino)-phenyl]-acetic acid or [2-(7-Chloro-quinolin-4-ylamino)-phenyl]-acetic acid methyl ester or when applicable, a pharmaceutically acceptable cationic salt counterion thereof or pharmaceutically acceptable acid addition salt thereof and is present in a concentration of from about 0.00001-5% w/v.

Various tonicity agents may be included in the compositions of the present invention to adjust tonicity, preferably to that of natural tears for ophthalmic compositions. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added. In general, however, the compositions will have one or more tonicity agents in a total concentration sufficient to cause the composition to have an osmolality of about 200-400 mOsm or equivalent to a sodium chloride solution between 0.7 and 1.1%.

An appropriate buffer system for ocular administration (e.g., sodium phosphate, sodium acetate, sodium citrate, sodium borate or boric acid) may be added to the compositions to prevent pH drift under storage conditions and to provide optimal pH for activity. The particular concentration will vary, depending on the agent employed.

Further, antioxidants may be added to pharmaceutical formulations for use in the methods of the invention to protect the active compound, e.g., [2-(7-Trifluoromethyl-quinolin-4-ylamino)-phenyl]-acetic acid, [2-(7-Trifluoromethyl-quinolin-4-ylamino)-phenyl]-acetic acid methyl ester, [2-(7-Chloro-quinolin-4-ylamino)-phenyl]-acetic acid or [2-(7-Chloro-quinolin-4-ylamino)-phenyl]-acetic acid methyl ester or when applicable, pharmaceutically acceptable cationic salt counterions thereof or pharmaceutically acceptable acid addition salts thereof, from oxidation during storage and/or or to provide antioxidant effects in the eye. Examples of such antioxidants include, but are not limited to, vitamin E and analogs thereof, ascorbic acid and derivatives, and butylated hydroxyanisole (BHA).

Additionally, preservatives may be added to the pharmaceutical formulation to protect active compounds described herein. Preservative agents are chemicals that prevent microbial growth and decomposition of a product. Examples are parabens such as methyl and propyl paraben, quarternary ammonium compounds such as benzalkonium chloride and phenoxyethyl alcohol.

### 5. Formulating Pharmaceutical Compositions For Ocular Administration

Exemplary pharmaceutical formulations for use in the methods of the invention may be prepared as follows. These exemplary formulations are provided for the purpose of illustrating the invention and should not be construed as limiting.

Batch quantities of polyoxyl 40 stearate, boric acid, sodium chloride, disodium edetate, and polyquaternium-1 are weighed and dissolved by stirring in 90% of the batch quantity of purified water. The pH is adjusted to 7.5±0.1 with NaOH and/or HCl. Under yellow light or reduced lighting, the batch quantity of the active compound as a stock solution in ethanol and the additional quantity of ethanol necessary for the batch are measured and added. Purified water is added quantity sufficient (q.s.) to 100%. The mixture is stirred for five minutes to homogenize and then filtered through a sterilizing filter membrane into a sterile recipient. Preferably, the above process is performed using glass, plastic or other non-metallic containers or containers lined with such materials.

### Formulation 1

| Ingredient | Amount (% w/v) |
|---|---|
| Active Compound | 0.00001-0.01 |
| Ethanol | 0.0505 |
| Polyoxyl 40 Stearate | 0.1 |
| Boric Acid | 0.25 |
| Sodium Chloride | 0.75 |
| Disodium Edetate | 0.01 |
| Polyquaternium-1 | 0.001 |
| NaOH/HCl | q.s., pH = 7.5 |
| Purified Water | q.s. 100% |

### Formulation 2

| Ingredient | Amount (% w/v) |
|---|---|
| Active Compound | 0.00001-0.01 |
| Polyoxyl 40 Stearate | 0.1 |
| Boric Acid | 0.25 |
| Sodium Chloride | 0.75 |
| Disodium Edetate | 0.01 |
| Polyquaternium-1 | 0.001 |
| NaOH/HCl | q.s., pH = 6.5-8 |
| Purified Water | q.s. 100% |

### Formulation 3

| Ingredient | Amount (% w/v) |
|---|---|
| Active Compound | 0.00001-0.01 |
| Polyoxyl 40 Stearate | 0.1 |
| Ethanol | 0.005-0.2 |
| Boric Acid | 0.25 |
| Sodium Chloride | 0.75 |
| NaOH/HCl | q.s., pH = 6.5-8 |
| Purified Water | q.s. 100% |

### Formulation 4

The following is an example of a formulation using an artificial tears carrier:

| Ingredient | Amount (% w/v) |
|---|---|
| Active Compound | 0.00001-0.01 |
| HPMC | 0.3 |
| Dextran 70 | 0.1 |
| Sodium Chloride | 0.8 |
| Potassium Chloride | 0.12 |
| Dibasic Sodium Phosphate | 0.025 |
| Disodium EDTA | 0.01 |
| Polyquaternium-1 | 0.001 + 10% excess |
| Purified Water | q.s. |
| NaOH/HCl | q.s. to pH 6-8 |

For each of the above formulations, the active compound is a compound having the formula:

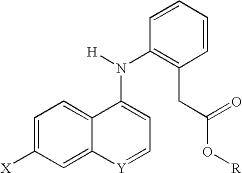

where X is H, F, Cl, Br, or CF₃; Y is N or N-oxide; and R is H, lower alkyl, lower alkoxyalkyl, or lower hydroxyalkyl. The compound may be a pharmaceutically acceptable acid addition salt, a pharmaceutically acceptable cationic salt counterion, a non-charged molecule or zwitterionic. In some embodiments, the active compound is [2-(7-Trifluoromethyl-quinolin-4-ylamino)-phenyl]-acetic acid, a pharmaceutically acceptable cationic salt counterion thereof, a pharmaceutically acceptable acid addition salt thereof or [2-(7-Trifluoromethyl-quinolin-4-ylamino)-phenyl]-acetic acid methyl ester or a pharmaceutically acceptable acid addition salt thereof. Other compounds useful in the above formulations include [2-(7-Chloro-quinolin-4-ylamino)-phenyl]-acetic acid, a pharmaceutically acceptable cationic salt counterion thereof, a pharmaceutically acceptable acid addition salt thereof or [2-(7-Chloro-quinolin-4-ylamino)-phenyl]-acetic acid methyl ester, a pharmaceutically acceptable acid addition salt thereof or a combination of such active compounds.

The following examples are provided for the purpose of illustrating the invention and should not be construed as limiting.

### EXAMPLE 1

### [2-(7-Trifluoromethyl-quinolin-4-ylamino)-phenyl]-acetic acid

This compound may be prepared by the method described in Example 38 of U.S. Pat. No. 3,637,710, the entire disclosure of which is hereby incorporated by reference.

The hydrochloride salt of this compound was prepared by the following alternative procedure:

To a suspension of 30 g of 5% palladium on carbon (50% wet) in 150 mL of methanol, was added a solution of 152 g of N,N-Dimethyl-2-(2-nitro-phenyl)-acetamide in 650 mL of methanol. Hydrogenation in a Parr apparatus was performed until a pressure drop corresponding to the theoretical amount of hydrogen was noted. The maximum pressure used was 20 psi. The reaction was fast and exothermic. The solution was cooled down to 22° C., filtered through celite and evaporated to give a solid. The product was dissolved in 180 mL of ether. The ether solution was dried (MgSO₄), filtered and evaporated to give 2-(2-Amino-phenyl)-N,N-dimethyl-acetamide as a yellow solid which was used directly in the next step.

To a refluxing solution of 124 g of 4-chloro-7-trifluoromethylquinoline and 41.4 mL of 4 M HCl/dioxane, in 625 mL of anhydrous acetonitrile, was added 124 g of 2-(2-Amino-phenyl)-N,N-dimethyl-acetamide in 175 mL of anhydrous acetonitrile over a 7 h period with mechanical stirring. The mixture was refluxed for another 4 h, cooled to 22° C. and left to stand overnight. The resulting hydrochloride salt was collected by filtration through a 600 mL sintered glass funnel, washed with ethyl acetate (200 mL) and then washed with a 3:1 solution of ethyl acetate/acetonitrile (3×200 mL). The hydrochloride salt was dissolved in water (3 L) and ethyl acetate was added (400 mL). The aqueous phase was washed with ethyl acetate (3×400 mL) and then neutralized to pH 7 by addition of 50% aqueous NaOH. A precipitate formed and the mixture was extracted with ethyl acetate (1×1.6 L then 2×200 mL). The organic solution was dried (MgSO₄, 34 g) and evaporated to give N,N-Dimethyl-2-[2-(7-trifluoromethyl-quinolin-4-ylamino)-phenyl]-acetamide as an off-white solid which was used directly in the next step. A small sample was recrystallized from hexane/ethyl acetate, M.P. 172-173° C.

A mixture of 163 g of N,N-Dimethyl-2-[2-(7-trifluoromethyl-quinolin-4-ylamino)-phenyl]-acetamide and 122.5 g of sodium hydroxide in 1630 mL of ethanol was refluxed for 5.5 h which resulted in a black solution. Normal phase silica TLC analysis (eluting with 1:1 ethyl acetate/hexane) indicated that all of the starting acetamide was consumed. About 1 L of the solvent was evaporated and then 3 L of water was added. About 2 L of the resulting solvent mixture was evaporated. The residual mixture was partitioned between water (4.75 L) and ether (1 L). The aqueous layer was washed with ether (3×1 L). The residual ether dissolved in the water was removed by evaporation under vacuum over a 45 minute period and the aqueous solution was then acidified with concentrated HCl to pH 4.50 over a 105 minute period. The resulting suspension was heated to 37° C. for 30 minutes then cooled with an ice bath for 1.25 h. The white suspension was filtered and washed with a solution of dilute HCl (pH=4.5; 2×500 mL). After drying under vacuum, the powder was triturated in 325 mL of refluxing methanol for 1.25 h and then cooled with an ice bath for 1.5 h. The mixture was filtered and washed with 250 mL of cold methanol (Note: the filtration was difficult). The solid was dried under vacuum and then gradually added to 4370 mL of 0.25 N HCl at a temperature of 60° C. The mixture was further heated to 95-100° C. which completely dissolved the solid. The hot solution was filtered through celite then allowed to stand at 22° C. overnight. The resulting suspension was further cooled with an ice bath for 2.5 h. The powder was collected and washed with cold 0.25 N HCl (2×500 mL) and then dried under vacuum at 60° C. [2-(7-Trifluoromethyl-quinolin-4-ylamino)-phenyl]-acetic acid hydrochloride was obtained as a yellowish powder; M.P. 103-104° C.

### EXAMPLE 2

[2-(7-Chloro-quinolin-4-ylamino)-phenyl]-acetic acid hydrochloride was prepared by the method outlined in Example 11 by substituting 4,7-dichloroquinoline for 4-chloro-7-trifluoromethylquinoline; m.p. 113-116° C.

### EXAMPLE 3

### Preparation of Other Active Compounds

[2-(7-Trifluoromethyl-quinolin-4-ylamino)-phenyl]-acetic acid methyl ester may be prepared by the procedure described in the '710 patent mentioned above as follows:

A mixture of 7.2 g of 4-chloro-7-trifluoromethylquinoline, 5.4 g of methyl 2-aminophenylacetate and 250 mL of acetonitrile is heated under reflux for 18 hours. The solvent is evaporated under reduced pressure and the residue extracted 3 times with 200 mL portions of hot water. The aqueous extract is rendered basic with 10% ammonium hydroxide whereupon the desired compound precipitated; it was recrystallized from aqueous ethanol yielding colorless needles with a melting point of 140-141° C.

[2-(7-Chloro-quinolin-4-ylamino)-phenyl]-acetic acid methyl ester may be prepared by the procedure described in the '710 patent as follows:

A mixture of 160 g of 4,7-dichloroquinoline, 80 g of methyl 2-aminophenylacetate and 500 mL of acetonitrile is heated at reflux temperature for 24 hours. The solvent is evaporated under reduced pressure and the residue extracted 4 times with 750 mL portions of hot water. The aqueous extract is rendered basic (pH 8) with 10% ammonium hydroxide whereupon [2-(7-Chloro-quinolin-4-ylamino)-phenyl]-acetic acid methyl ester separates as an oil which crystallizes upon addition of ethanol. The product may be recrystallized from aqueous methanol, M.P. 147-148° C.

The hydrochloride of [2-(7-Chloro-quinolin-4-ylamino)-phenyl]-acetic acid methyl ester may be prepared in conventional manner by treating a solution of the free base in ether with gaseous hydrochloric acid; M.P. 224-226° C.

To a solution of 6 g of [2-(7-Chloro-quinolin-4-ylamino)-phenyl]-acetic acid methyl ester in 100 mL of hot methanol is added 3 g of methanesulfonic acid. The solution is cooled to room temperature and 750 mL of ether is added. The product precipitates as off-white crystals and is collected by filtration. The resulting methanesulfonate of the above-mentioned compound is recrystallized from methanol-ether, M.P. 179-181° C.

[2-(7-Chloro-quinolin-4-ylamino)-phenyl]-acetic acid may be prepared by the procedure described in the '710 patent as follows:

A mixture of 8.1 g of [2-(7-Chloro-quinolin-4-ylamino)-phenyl]-acetic acid methyl ester, 150 mL of 1 N sodium hydroxide and 25 mL of methanol is refluxed for 30 minutes. The methanol is distilled off, the aqueous solution is treated with charcoal, filtered and cooled to room temperature. To dissolve the sodium salt of the product, 150 mL of water is added. Addition of 3 N hydrochloric acid until pH 5 is reached causes precipitation of [2-(7-Chloro-quinolin-4-ylamino)-phenyl]-acetic acid. The product may be collected by filtration and washed well with water, ethanol and ether, M.P. 259-260° C.

[2-(7-Chloro-quinolin-4-ylamino)-phenyl]-acetic acid ethyl ester may be prepared by the procedure described in the '710 patent as follows:

A mixture of 50 g of 4,7-dichloroquinoline, 43 g of 5 ethyl 2-aminophenylacetate, 450 mL of acetonitrile and 50 mL of dimethylsulfoxide is heated at reflux temperature for 18 hours. The solvent is evaporated under reduced pressure and the residue extracted 4 times with 500 mL portions of hot water. The aqueous extract is cooled to 40° C. and rendered basic to pH 8 with 10% ammonium hydroxide whereupon [2-(7-chloro-quinolin-4-ylamino)-phenyl]-acetic acid ethyl ester separates as an oil. The supernatant liquid is decanted and the oil dissolved in chloroform and dried over magnesium sulfate. The solvent is evaporated under reduced pressure and the residue dissolved in 10 mL of chloroform and chromatographed on a neutral alumina column using chloroform as the eluent. The solvent is evaporated under reduced pressure leaving a yellow oil which crystallizes on standing. The product may be recrystallized from a mixture of ethanol and ether, M.P. 107-109° C.

[2-(7-Chloro-quinolin-4-ylamino)-phenyl]-acetic acid n-butyl ester hydrochloride may be prepared by the procedure described in the '710 patent as follows:

A mixture of 250 g of 2-nitrophenylacetic acid, 500 mL of n-butanol and 10 mL of concentrated sulfuric acid is heated at reflux temperature for 18 hours. Upon cooling, the solution is diluted with 1000 mL of water and is rendered basic with 10% ammonium hydroxide. The product separates as an oil which is extracted into 1000 mL (2×500 mL) of ether. The combined ethereal extracts are washed well with water and the ether removed under reduced pressure to yield n-butyl 2-nitrophenylacetate, B.P. 119° C./0.15 mm.

A mixture of 50.3 g of butyl 2-nitrophenylacetate, 5 g of 5% palladium on barium sulfate and 300 mL of ethylacetate is hydrogenated at atmospheric pressure until 3 mole equivalents of hydrogen is absorbed. The catalyst is separated by filtration and the solvent is removed under reduced pressure to yield n-butyl 2-aminophenylacetate. This material may be used without further purification.

A mixture of 40 g of 4,7-dichloroquinoline, 1.7 mL of concentrated hydrochloric acid and 250 mL of acetonitrile is heated to 65° C. To the solution, 43.5 g of n-butyl 2-aminophenylacetate is added. The reaction temperature is lowered to 45° C. and maintained at this temperature for 18 hours. [2-(7-Chloro-quinolin-4-ylamino)-phenyl]-acetic acid n-butyl ester hydrochloride separates and is collected by filtration and washed with acetonitrile. The product may be recrystallized from a mixture of methanol and acetone, M.P. 167-168° C.

[2-(7-Chloro-quinolin-4-ylamino)-phenyl]-acetic acid 2,3-dihydroxy-propyl ester may be prepared by the procedure described in the '710 patent as follows:

50 mg of sodium is dissolved in 150 mL of isopropylidene glycerol and 6.9 g of [2-(7-Chloro-quinolin-4-ylamino)-phenyl]-acetic acid methyl ester added. The reaction is heated to 125° C. for 3 hours under a nitrogen atmosphere, and then cooled and poured into ice-water. The aqueous phase is extracted with chloroform and the combined chloroform extracts are dried over magnesium sulfate and the chloroform removed under reduced pressure to yield a yellow oil. The oil is dissolved in a minimum of chloroform and chromatographed on a neutral alumina column using chloroform as an eluant. The chloroform is removed under reduced pressure to yield an oil which is heated with 2 N hydrochloric acid for 10 minutes. The aqueous solution is rendered basic with 10% ammonium hydroxide and extracted with chloroform. The chloroform extracts are dried over magnesium sulfate and the chloroform removed under reduced pressure to yield an oil which crystallizes on trituration with petrol-ether and is collected by filtration, M.P. 157-159° C.

[2-(7-Chloro-1-oxy-quinolin-4-ylamino)-phenyl]-acetic acid methyl ester may be prepared by the procedure described in the '710 patent as follows:

A solution of 10 g of [2-(7-Chloro-quinolin-4-ylamino)-phenyl]-acetic acid methyl ester in 100 mL of chloroform is added to a mixture of 150 mL of chloroform and 4.5 g of 85% m-chloroperbenzoic acid. The color of the solution immediately turns a greenish-yellow. The solution is then heated under reflux on a steam bath for 15 minutes and during this time the color of the solution changes to a deep reddish-yellow. The reaction mixture is then cooled to room temperature and the solution is extracted with sodium bicarbonate 250 mL washed with water (4×250 mL) and dried over magnesium sulfate. The chloroform is removed under reduced pressure and the residue is dissolved in a minimum of hot ethanol. The ethanol solution is then added to a large volume of petroleum ether (circa 2L) and stirred for 18 hours; the product crystallizes on the walls of the flask. This procedure is repeated once more to yield a yellow powder, M.P. 204-205° C. U.V. Absorption shows λₘₐₓ at 388 μm, which is characteristic of quinoline-1-oxides.

[2-(7-Trifluoromethyl-1-oxy-quinolin-4-ylamino)-phenyl]-acetic acid methyl ester may be prepared by the procedure described in the '710 patent as follows:

A solution of 13.4 g of [2-(7-Trifluoromethyl-quinolin-4-ylamino)-phenyl]-acetic acid methyl ester in 250 mL of chloroform is added to a mixture of 200 mL of chloroform and 10.2 g of 85% m-chloroperbenzoic acid. The solution is then heated under reflux for 40 minutes. The reaction mixture is cooled to room temperature and the solution is extracted with 5% sodium bicarbonate (3×500 mL), washed with water (2×500 mL) and dried over magnesium sulfate. The chloroform solution is concentrated under reduced pressure to about 25 mL and chromatographed on a neutral alumina column using a 10/90 mixture of methanol-chloroform as the eluant. The chloroform is evaporated to dryness under reduced pressure and the residue is recrystallized from a mixture of acetone and methanol, M.P. 209-211° C.

### EXAMPLE 4

[2-(7-Trifluoromethyl-quinolin-4-ylamino)-phenyl]-acetic acid sodium salt was prepared as follows:

To a solution of 4.12 g of [2-(7-Trifluoromethyl-quinolin-4-ylamino)-phenyl]-acetic acid methyl ester in 150 mL of methanol was added 12.0 mL of 1 M aqueous NaoH. The solution was refluxed for 40 minutes and the solvents were evaporated. The residue was mixed with acetone then evaporated again. The residue was then dissolved in 40 mL of boiling acetone with addition of a few mL of water to dissolve. The hot solution was filtered then cooled and left at ca. 3° C. overnight. The resulting white solid was collected and washed with cold acetone to give 3.03 g of [2-(7-Trifluoromethyl-quinolin-4-ylamino)-phenyl]-acetic acid sodium salt; MS found M+H=347.

### EXAMPLE 5

### Use of [2-(7-Trifluoromethyl-quinolin-4-ylamino)-phenyl]-acetic acid to treat skin inflammatory conditions

A pharmaceutically acceptable topical formulation of [2-(7-Trifluoromethyl-quinolin-4-ylamino)-phenyl]-acetic acid hydrochloride may be administered topically to the surface of the skin of a patient, in order to treat skin inflammatory conditions such as psoriasis, psoriatic arthritis, or atopic dermatitis.

### EXAMPLE 6

### Use of [2-(7-Trifluoromethyl-quinolin-4-ylamino)-phenyl]-acetic acid to treat arthritides

A pharmaceutically acceptable topical formulation of [2-(7-Trifluoromethyl-quinolin-4-ylamino)-phenyl]-acetic acid hydrochloride may be administered topically to the surface of the skin of a patient, in order to treat arthritides such as rheumatoid arthritis or osteoarthritis.

### 6. Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All references, patents, patent application publications, and patent applications cited herein are hereby incorporated by reference to the same extent as if each of these references, patents, patent application publications, and patent applications were separately incorporated by reference herein.

What is claimed is:

1. A method of treating a skin disorder, the method comprising topically administering a therapeutically effective amount of a pharmaceutically acceptable formulation to a patient in need thereof, wherein the pharmaceutically acceptable formulation comprises an active compound having the formula: 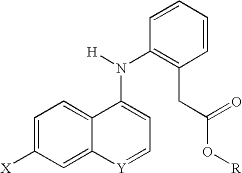
wherein
X is H, F, Cl, Br or CF₃;
Y is N, N-oxide (N—O), or a pharmaceutically acceptable acid addition salt thereof; and
R is H, lower alkyl, lower alkoxyalkyl, lower hydroxyalkyl, or a pharmaceutically acceptable cationic salt counterion.

2. The method of claim 1, wherein the step of topically administering comprises transdermal administration.

3. The method of claim 1, wherein the skin disorder is at least one of basal cell carcinoma, cutaneous metastatic breast cancer, pima squamous cell tumor, metastatic melanoma in the skin, malignancy or tumor in the skin, genital wart, corn on a foot, actinic keratosis, psoriasis, psoriatic arthritis, atopic dermatitis, liver spot, fungal lesion, skin lesion, or hair loss during pregnancy.

4. The method of claim 3, wherein the skin disorder is psoriasis.

5. The method of claim 3, wherein the skin disorder is atopic dermatitis.

6. The method of claim 3, wherein the skin disorder is actinic keratosis.

7. The method of claim 3, wherein the skin disorder is psoriatic arthritis.

8. The method of claim 1 wherein X is CF₃; Y is N or a pharmaceutically acceptable acid addition salt thereof; and R is H or a pharmaceutically acceptable cationic salt counterion.

9. The method of claim 1 wherein X is Cl; Y is N or a pharmaceutically acceptable acid addition salt thereof; and R is H or a pharmaceutically acceptable cationic salt counterion.

10. A method of treating a disorder associated with pain, fever, or inflammation, the method comprising topically administering a therapeutically effective amount of a pharmaceutically acceptable formulation to a patient in need thereof, wherein the pharmaceutically acceptable formulation comprises an active compound having the formula: 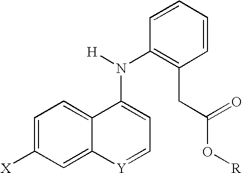
wherein
X is H, F, Cl, Br or CF₃;
Y is N, N-oxide (N—O), or a pharmaceutically acceptable acid addition salt thereof; and
R is H, lower alkyl, lower alkoxyalkyl, lower hydroxyalkyl, or a pharmaceutically acceptable cationic salt counterion.

11. The method of claim 10, wherein the disorder is at least one of rheumatic fever, lower back or neck pain, dysmenorrhea, sprain or strain, headache, toothache, myositis, neuralgia, synovitis, rheumatoid arthritis, degenerative joint diseases, osteoarthritis, gout and ankylosing spondylitis, bursitis, bums, post-surgical or dental procedures, or pain or inflammation associated with herpes vesicles.

12. The method of claim 10, wherein the disorder is rheumatoid arthritis.

13. The method of claim 10, wherein the disorder is osteoarthritis.

14. The method of claim 10, wherein the disorder is bursitis.

15. The method of claim 10 wherein X is CF₃; Y is N or a pharmaceutically acceptable acid addition salt thereof; and R is H or a pharmaceutically acceptable cationic salt counterion.

16. The method of claim 10 wherein X is Cl; Y is N or a pharmaceutically acceptable acid addition salt thereof; and R is H or a pharmaceutically acceptable cationic salt counterion.

17. A method of treating a proliferative disorder, the method comprising topically administering a therapeutically effective amount of a pharmaceutically acceptable formulation to a patient in need thereof, wherein the pharmaceutically acceptable formulation comprises an active compound having the formula: 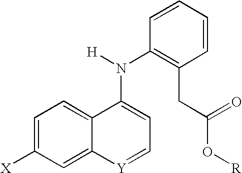
wherein
X is H, F, Cl, Br or CF₃;
Y is N, N-oxide (N—O), or a pharmaceutically acceptable acid addition salt thereof; and
R is H, lower alkyl, lower alkoxyalkyl, lower hydroxyalkyl, or a pharmaceutically acceptable cationic salt counterion.

18. The method of claim 17, wherein the proliferative disorder is at least one of diabetic retinopathy or tumor angiogenesis.

19. The method of claim 17, wherein the step of topically administering comprises transdermal or ocular administration.

20. The method of claim 13 wherein X is CF₃; Y is N or a pharmaceutically acceptable acid addition salt thereof; and R is H or a pharmaceutically acceptable cationic salt counterion.

21. The method of claim 13 wherein X is Cl; Y is N or a pharmaceutically acceptable acid addition salt thereof; and R is H or a pharmaceutically acceptable cationic salt counterion.

22. A method of treating an ocular disorder, the method comprising topically administering a therapeutically effective amount of a pharmaceutically acceptable formulation to a patient in need thereof, wherein the pharmaceutically acceptable formulation comprises an active compound having the formula: 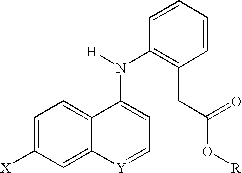
wherein
X is H, F, Cl, Br or CF₃;
Y is N, N-oxide (N—O), or a pharmaceutically acceptable acid addition salt thereof; and
R is H, lower alkyl, lower alkoxyalkyl, lower hydroxyalkyl, or a pharmaceutically acceptable cationic salt counterion.

23. The method of claim 22 wherein X is CF₃; Y is N or a pharmaceutically acceptable acid addition salt thereof; and R is H or a pharmaceutically acceptable cationic salt counterion.

24. The method of claim 22 wherein X is Cl; Y is N or a pharmaceutically acceptable acid addition salt thereof; and R is H or a pharmaceutically acceptable cationic salt counterion.

25. The method of claim 22, further comprising the step of administering an antimicrobial or antiviral agent.
